(19) United States
(12) Patent Application Publication
(10) Pub. No.: US 2021/0322332 A1
(43) Pub. Date: Oct. 21, 2021

(54) STATIN-ENCAPSULATED NANOPARTICLE PREPARATION, DENTAL PULP-DERIVED STEM CELLS CONTAINING SAME, AND CELL PREPARATION CONTAINING SUCH STEM CELLS

(71) Applicant: Masaaki Il, Hyogo (JP)

(72) Inventors: Masaaki II, Osaka (JP); Toshiro HAMAZONO, Tokyo (JP)

(21) Appl. No.: 17/054,265

(22) PCT Filed: May 10, 2019
(86) PCT No.: PCT/JP2019/018844

§ 371 Date: Nov. 10, 2020

(30) Foreign Application Priority Data
May 10, 2018 (JP) 2018-091722

(51) Int. Cl. A61K 9/51; A61K 35/28; A61K 31/366

(52) U.S. Cl. CPC A61K 9/5153; A61K 31/366; A61K 35/28

(57) ABSTRACT

The present invention pertains to a technology for improving the function of stem cells used as cell preparations and enables the improvement of therapeutic effects on diseases. The present invention is a statin-encapsulated nanoparticle preparation which is for enhancing the function of dental pulp-derived stem cells, and which contains statin-encapsulated nanoparticles in which a statin is encapsulated in nanoparticles that include a bioabsorbable polymer.

### TECHNICAL FIELD

The present invention relates to a statin-encapsulated nanoparticle preparation, a dental pulp-derived stem cell containing the same, and a cell preparation containing the same

### BACKGROUND ART

Statin is known as a compound which inhibits HMG-CoA reductase which is a rate-limiting enzyme of cholesterol biosynthesis in the liver. Statin can reduce the cholesterol level in blood and is thus used in therapeutic agents for hypercholesterolemia. Moreover, in addition to hypercholesterolemia, clinical tests have revealed that statin is also effective to ischemic heart diseases, such as angina pectoris and myocardial infarction, and diseases, such as arteriosclerosis, due to the anti-inflammatory activity of the statin.

Various studies have been conducted to improve the therapeutic effect of statin on the above-described diseases and to reduce side effects caused by statin. For example, Patent Literature 1 discloses that in a case of administration of statin for acceleration of neovascularization, the statin is included in nanoparticles, and the statin-included nanoparticles are topically administered to patients, thereby enabling the acceleration of the neovascularization with a fewer amount of statin than before.

As described above, statin exhibits various activities and in particular, has anti-inflammatory activity, and therefore, application of the statin to inflammatory diseases has been actively studied. For example, Non-Patent Literature 1 discloses that simvastatin which is a type of statin exhibits anti-inflammatory activity in a mouse inflammatory bowel disease model. Moreover, Non-Patent Literature 2 describes an anti-inflammatory effect of atorvastatin on patients suffering from Crohn's disease.

Moreover, in recent years, studies of treating various diseases with pluripotent stem cells have been conducted. Examples of stem cells generally include embryonic stem cells (ES cells) and mesenchymal somatic stem cells such as bone marrow-derived stem cells and adipose derived stem cells, and additionally, induced pluripotent stem cells (iPS cells) and the like, and such cells are adopted in various studies. Among them, the study of adipose derived stem cells is rapidly developing, and clinical tests of regenerative medicine for various diseases are widely performed. For example, Non-Patent Literature 3 discloses that stem cells derived from adipose tissue are directly administered to the myocardium of a myocardial infarction model mouse, thereby improving the function of the heart and reducing the infarct size.

It is also reported that adipose derived stem cells exhibit an enteritis depression effect in a drug-induced enteritis mouse model in addition to use in the regenerative medicine (for example, see Non-Patent Literature 4).

### CITATION LIST

### Patent Literature

[Patent Literature 1] Japanese Laid-Open Patent Publication No. 2012-21002

### Non-Patent Literature

[Non-Patent Literature 1] Yosuke, Abe et al., Ucer, 37(2010), 169-173.
[Non-Patent Literature 2] Grip, O et al, Br J Pharmacol. 155(2008), 1085-1092.
[Non-Patent Literature 3] Masaaki Ii et al., Laboratory Investigation (2011) 91, 539-552
[Non-Patent Literature 4] Gonzalez, M A et al. Gastroenterology 136(2009), 978-989.

### SUMMARY OF INVENTION

### Technical Problem

In order to treat ischemic heart disease such as myocardial infarction, for example, in the case of administering statin-encapsulated nanoparticles as disclosed in Non-Patent Literature 1, it is recognized that lower doses of statins than ever before is effective by locally administering statin-encapsulated nanoparticles to a patient, but topical administration to the affected part is required, and administration is not easy, and further improvement of effectiveness is also desired. Further, when intravenous administration or the like is used without using local administration, more dosage is required, and side effects may occur.

On the other hand, in the case of treating ischemic heart disease such as myocardial infarction by using stem cells as disclosed in Non-Patent Literature 3, topical administration of stem cells to the affected part is required, and, for example when administered to the myocardium of a mouse, an amount of 5×10⁵ cells/mouse is needed to obtain the therapeutic effect. Somatic stem cells derived from mesenchymal tissue can be used as stem cells, and such stem cells can be obtained from bone marrow or adipose tissue, but the number of cells which is obtainable onetime is limited. Therefore, when stem cells are used for the treatment or the like, it is preferable to use a small number of cells. Thus, in order to obtain a remarkable therapeutic effect by a fewer number of stem cells, it is necessary to improve the various functions of the stem cells.

In order to make it more efficiently to the effect of statin which is exhibited in treatment of inflammatory diseases by using the statin as disclosed in, for example, Non-Patent Literatures 1 and 2, the statin may be included in nanoparticles to obtain statin-included nanoparticles, which may be administered to patients as disclosed in, for example, Patent Literature 1. However, in Patent Literature 1, the statin-included nanoparticles are topically administered to patients. Thus, the effectiveness is confirmed with a smaller amount of the statin than before, but since the administered statin nanoparticles are, for example, phagocytized by macrophages, they are likely to be non-uniformly distributed in lesions, and therefore a stable therapeutic effect is hardly obtained.

Meanwhile, also when inflammatory diseases are treated with only stem cells as disclosed in Non-Patent Literature 4, topical administration of the stem cells to a diseased portion is required, and an enormous volume of cells are required. Therefore, not only cost and time are required, but also the frequency of occurrence of side effect due to cell administration increases. Moreover, when autologous cell transplant is assumed, in a case where the number of adipose tissues is small and thereby the number of separable stem cells is small or in a case where the stem cell function is degraded due to factors of turnover diseases such as an advanced age and/or diabetes mellitus, various types of functions of the stem cells have to be improved in order to obtain remarkable therapeutic effects from a small number of stem cells.

The present invention has been made in view of the aforementioned problems, and it is an object of the present invention to improve the function of stem cells used as a cell preparation and to improve the therapeutic effect on diseases.

### Solution to Problem

As a result of intensive studies, the inventors have found that the function of stem cells is enhanced and statin can be efficiently delivered to a desired lesion by incorporating statin-encapsulated nanoparticles constituting of statin encapsulated in nanoparticles into pulp-derived stem cells, and it is found that a high effect is exhibited in the treatment of various diseases such as ischemic diseases and inflammatory diseases.

That is, the present invention is as follows:

In one aspect of the present invention, the invention relates to

[1] a statin-encapsulated nanoparticle preparation for enhancing a function of a dental pulp-derived stem cell comprising: a nanoparticle containing a bioabsorbable polymer; and statin included in the nanoparticle.

The statin-encapsulated nanoparticle preparation according to the present invention can enhance the function of the treated dental pulp-derived stem cell when a dental pulp-derived stem cell is treated with the statin-encapsulated nanoparticle preparation, and administering the dental pulp-derived stem cell into a living body exhibits various effectiveness. Specifically, when stem cells are treated with the statin-encapsulated nanoparticle preparation according to the present invention, the treated dental pulp-derived stem cells capture the statin-encapsulated nanoparticles by fagocytosis, and the dental pulp-derived stem cells incorporating the statin-encapsulated nanoparticles have enhanced immunosuppression ability in addition to enhancing migration ability and an ability to produce an angiogenesis factor. Thus, when a dental pulp-derived stem cell which is treated with statin-encapsulated nanoparticles according to the present invention is administered to the body of a patient suffering from various diseases, it exhibits various remarkable disease therapeutic effects by the action of the enhanced function of the stem cells and the action of statin gradually released from the stem cells.

In one embodiment of the statin-encapsulated nanoparticle preparation of the present invention,

[2] the bioabsorbable polymer comprise a polylactic acid polymer (polylactic acid: PLA) and/or a poly(lactic-co-glycolic acid) (PLGA) copolymer.

PLA and PLGA can release the encapsulated statin by being hydrolyzed in the body. In addition, PLA is decomposed into lactic acid by hydrolysis, and PLGA is decomposed into lactic acid and glycol by hydrolysis. PLA and PLGA are finally decomposed into water and carbon dioxide which is harmless to animals such as human, so that PLA or PLGA is preferably used as a nanoparticle material.

In one embodiment of the statin-encapsulated nanoparticle preparation of the present invention,

[3] the enhanced function of the dental pulp-derived stem cell is at least one of a migration ability and an ability to produce an angiagenic factor.

In one embodiment of the statin-encapsulated nanoparticle preparation of the present invention,

[4] the statin-encapsulated nanoparticle preparation according to [1] or [2], wherein the enhancement of the function of the dental pulp-derived stem cell is an increase in expression of HGF.

In another aspect of the present invention, the invention relates to

[5] a statin-encapsulated nanoparticle preparation comprising: a nanoparticle containing a bioabsorbable polymer; and statin included in the nanoparticle, wherein the preparation is for enhancing the therapeutic effect of a cell preparation containing a dental pulp-derived stem cell for treating an ischemic disease, an inflammatory disease, or a neurodegenerative disease.

In another aspect of the present invention, the invention relates to

[6] a statin-encapsulated nanoparticle preparation comprising: a nanoparticle containing a bioabsorbable polymer; and statin included in the nanoparticle, wherein the preparation is for enhancing the therapeutic effect of a cell preparation containing a dental pulp-derived stem cell or adipose-derived stem cell for treating an inflammatory disease or a neurodegenerative disease.

In another aspect of the present invention, the invention relates to

[7] a dental pulp-derived stem cell containing the statin-encapsulated nanoparticle preparation according to any one of the above [1] to [3].

Since the dental pulp-derived stem cell according to the present invention contains the statin-encapsulated nanoparticle, the cell function is enhanced by the statin-encapsulated nanoparticle, and the dental pulp-derived stem cell has the superior effect in the treatment of various diseases including ischemic diseases.

In another aspect of the present invention, the invention relates to

[8] a cell preparation for treating ischemic diseases, comprising the dental pulp-derived stem cells described in [7] above.

In another aspect of the present invention, the invention relates to

[9] a cell preparation for treating inflammatory diseases, comprising dental pulp-derived stem cells described in [7] above.

In another aspect of the present invention, the invention relates to

[10] a cell preparation for treating neurodegenerative diseases, comprising dental pulp-derived stem cells described in [7] above.

In another aspect of the present invention, the invention relates to

[11] the cell preparation according to any one of [7] to [10], wherein the cell preparation is characterized in being used for intravenous administration, intravenous administration, or topical administration.

The dental pulp-derived stem cells according to the present invention can be formulated as a cell preparation by mixing with a pharmaceutically acceptable solvent and an excipient. It is preferable that the administration of the stem cell to the living body does not require surgery such as abdominal opening, and it is preferable that the stem cell is formulated as a cell preparation for intravenous administration or intraarterial administration. Thus, the stem cell according to the present invention can be simply administered to a patient. Since the function of the stem cell is enhanced, a superior effect can be obtained with a small dose even in intravenous administration or intra-arterial administration, not in local administration. In addition, the function-enhanced stem cell preparation according to the present invention is capable of accumulating stem cells in an inflammatory part such as an intestine by administering intra-arterial administration, which is difficult to reach by intravenous administration. Thus, it is not necessary to perform local administration, and by using intra-arterial administration, cells can be uniformly distributed to a lesion with a small dose, and a stable high effect can be obtained.

In another aspect of the present invention, the invention relates to

[12] a cell preparation for treating diseases related to neuroinflammation, the cell preparation comprising a dental pulp-derived stem cell.

In another aspect of the present invention, the invention relates to

[13] a method for treating a subject suffering from ischemic disease, the method comprising administering a cell preparation comprising the dental pulp-derived stem cell according to the above [7] to the subject.

In another aspect of the present invention, the invention relates to

[14] a method for treating a subject suffering from inflammatory diseases, the method comprising administering a cell preparation comprising the dental pulp-derived stem cell according to above [7] to the subject.

In another aspect of the present invention, the invention relates to

[15] a method for treating a subject suffering from neurodegenerative diseases, the method comprising administering a cell preparation comprising the dental pulp-derived stem cell according to above [7] to the subject.

In another aspect of the present invention, the invention relates to

[16] a cell preparation comprising a dental pulp-derived stem cell according to [7], the cell preparation being used for treating a subject suffering from ischemic diseases.

In another aspect of the present invention, the invention relates to

[17] a cell preparation comprising a dental pulp-derived stem cell according to [7], the cell preparation being used for treating a subject suffering from inflammatory diseases.

In another aspect of the present invention, the invention relates to

[18] a cell preparation comprising the dental pulp-derived stem cell according to [7], the cell preparation being used for treating a subject suffering from neurodegenerative disease.

In another aspect of the present invention, the invention relates to

[19] use of a cell preparation comprising a dental pulp-derived stem cell according to [7] in producing a therapeutic agent for an ischemic disease.

In another aspect of the present invention, the invention relates to

[20] use of a cell preparation comprising the dental pulp-derived stem cell according to [7] in producing a therapeutic agent for an inflammatory disease.

In another aspect of the present invention, the invention relates to

[21] use of a cell preparation comprising a dental pulp-derived stem cell according to [7] in producing a therapeutic agent for a neurodegenerative disease.

### Effect of the Invention

According to the statin-encapsulated nanoparticle preparation, a dental pulp-derived stem cell comprising the same, and a cell preparation comprising the same of the present invention, the function of the dental pulp-derived stem cell can be enhanced, and when the stem cell is administered in a living body, it can bring a superior therapeutic effect on various diseases.

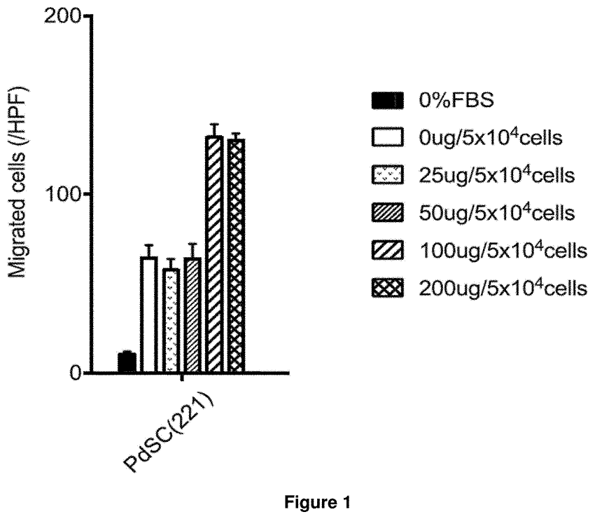

FIG. 1 is a graph showing measurement results of migration of dental pulp-derived stem cells treated with statin-encapsulated nanoparticles.

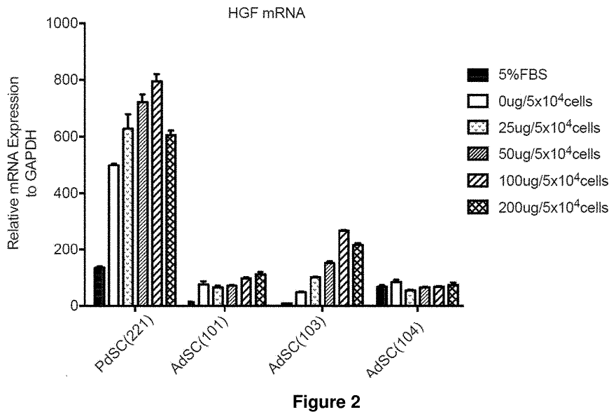

FIG. 2 is a graph showing measurement results of HGF expression levels of dental pulp-derived stem cells and adipose-derived stem cells treated with statin-encapsulated nanoparticles.

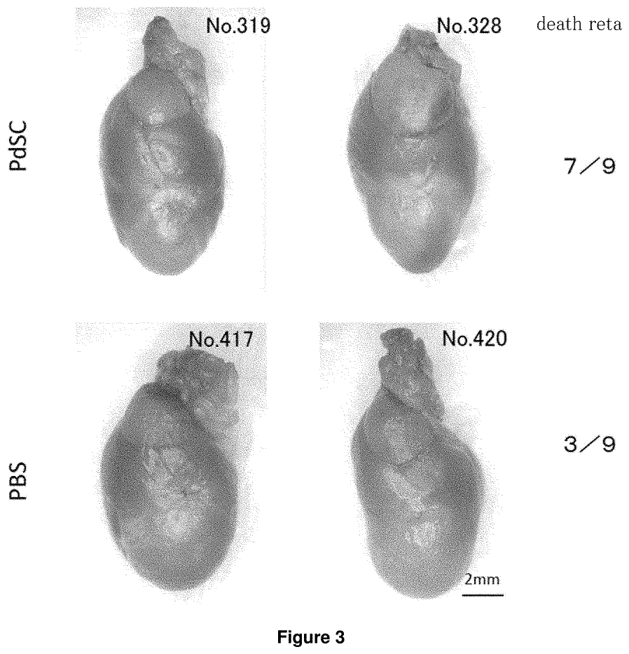

FIG. 3 is a photograph showing the heart of a myocardial infarction model mouse after 53 days from administering PBS or dental pulp-derived stem cells.

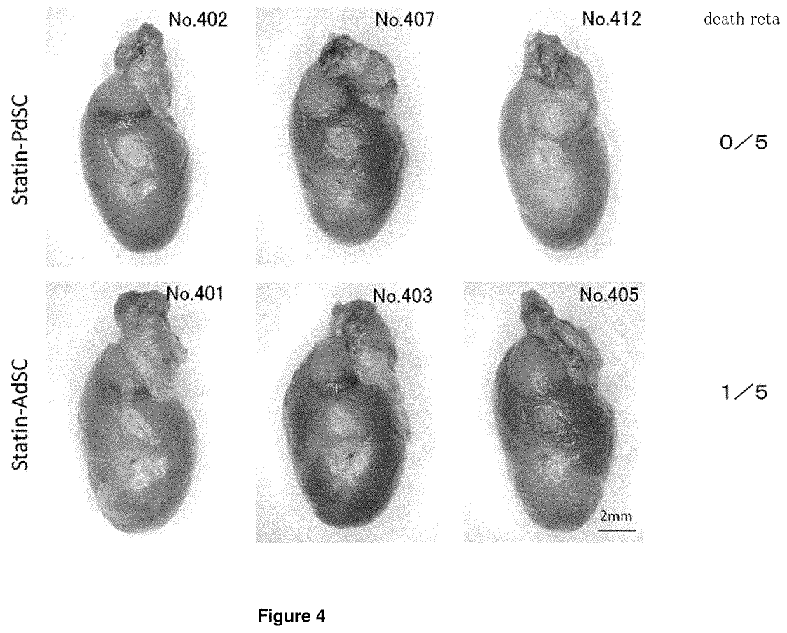

FIG. 4 is a photograph showing the heart of a myocardial infarction model mouse after 53 days from administering dental pulp-derived stem cells or adipose-derived stem cells comprising statin-encapsulated nanoparticles.

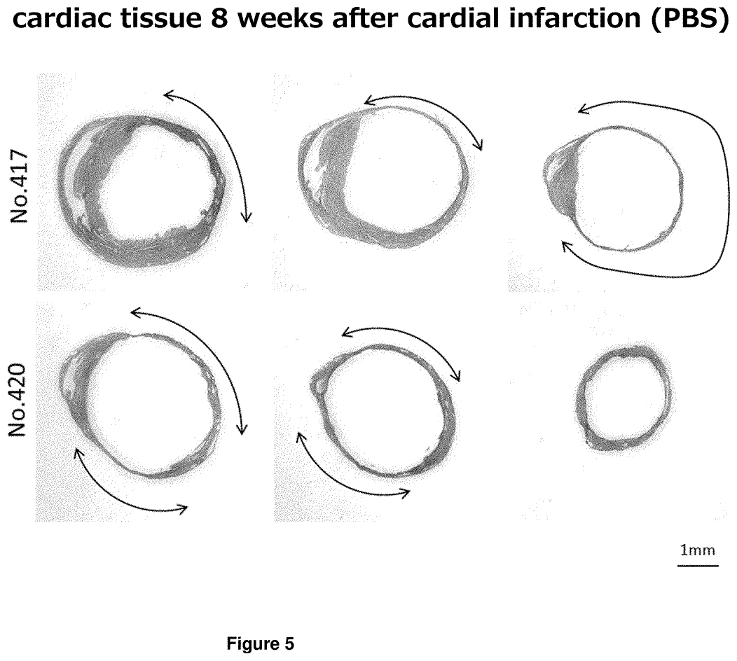

FIG. 5 is a photograph showing the result of trichrome staining of an infarct section of a heart from a myocardial infarction model mouse into which PBS is administered.

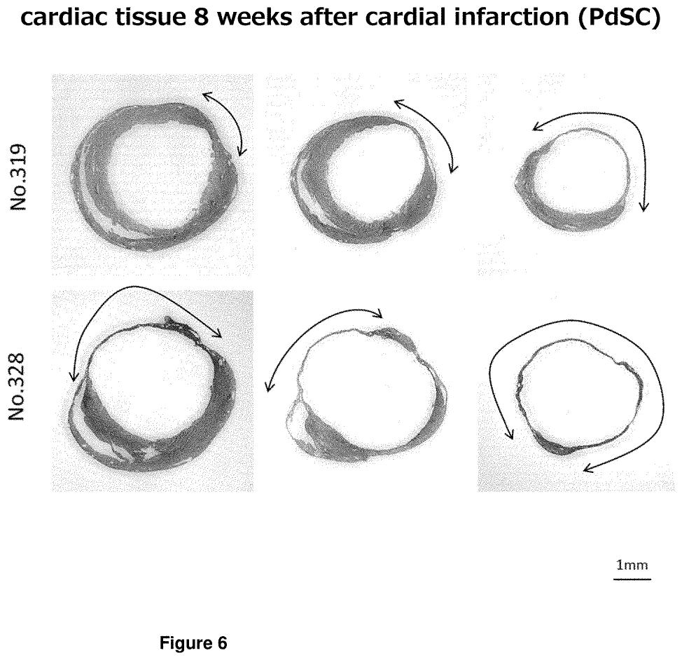

FIG. 6 is a photograph showing the result of trichrome staining of an infarct section of a heart from a myocardial infarction model mouse into which dental pulp-derived stem cells are administered.

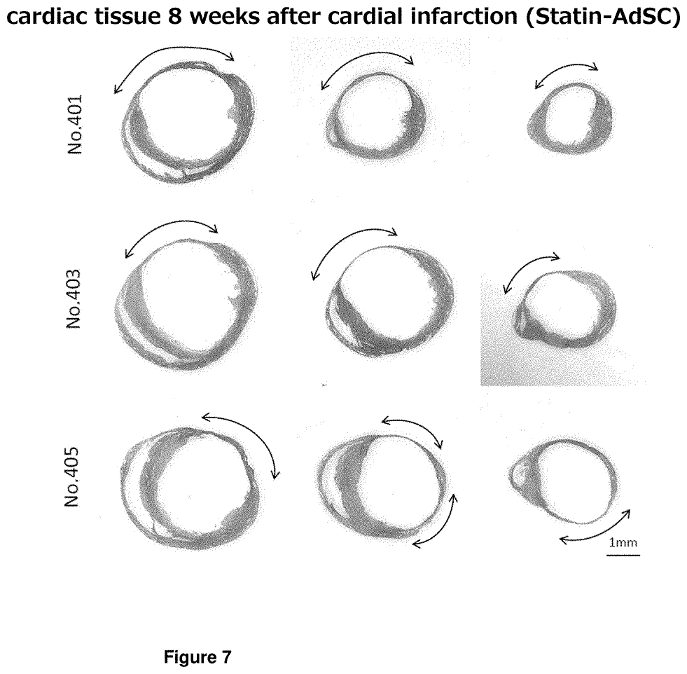

FIG. 7 is a photograph showing the result of trichrome staining of an infarct section of a heart from a myocardial infarction model mouse into which adipose-derived stem cells comprising statin-encapsulated nanoparticles are administered.

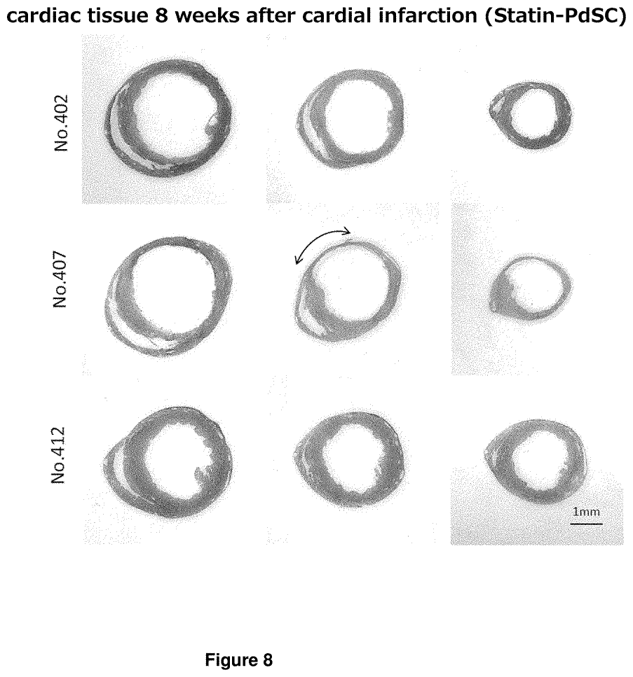

FIG. 8 is a photograph showing the result of trichrome staining of an infarct section of a heart from a myocardial infarction model mouse into which dental pulp-derived stem cells comprising statin-encapsulated nanoparticles are administered.

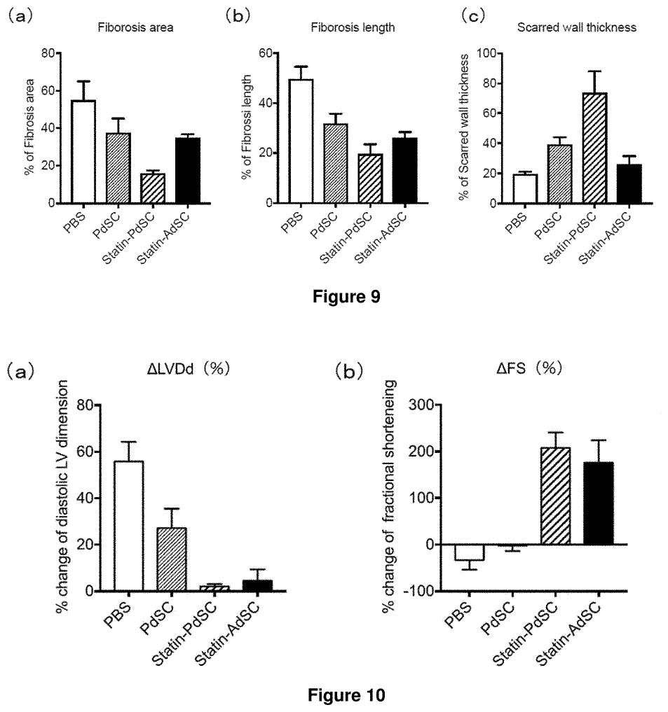

FIG. 9(A) is a graph showing the area of a fiberized portion in a section of an infarct portion of a heart from a myocardial infarction model mouse into which PBS, dental pulp-derived stem cells, adipose-derived stem cells comprising statin-encapsulated nanoparticles, or dental pulp stem cells comprising statin-encapsulated nanoparticles was administered, and (B) is a graph showing the length of a fiberized portion in the sections, and (C) is a graph showing the thickness of the myocardial wall of the fiberized portion in each section.

FIG. 10 is a graph showing a result of ultrasonic diagnosis of the heart of a myocardial infarction model mouse into which PBS, dental pulp-derived stem cells, adipose-derived stem cells comprising statin-encapsulated nanoparticles, or dental pulp stem cells comprising statin-encapsulated nanoparticles was administered; and (A) shows a left ventricular end-diastolic diameter (LVDD), and (B) shows a left ventricular fractional shortening rate (FS).

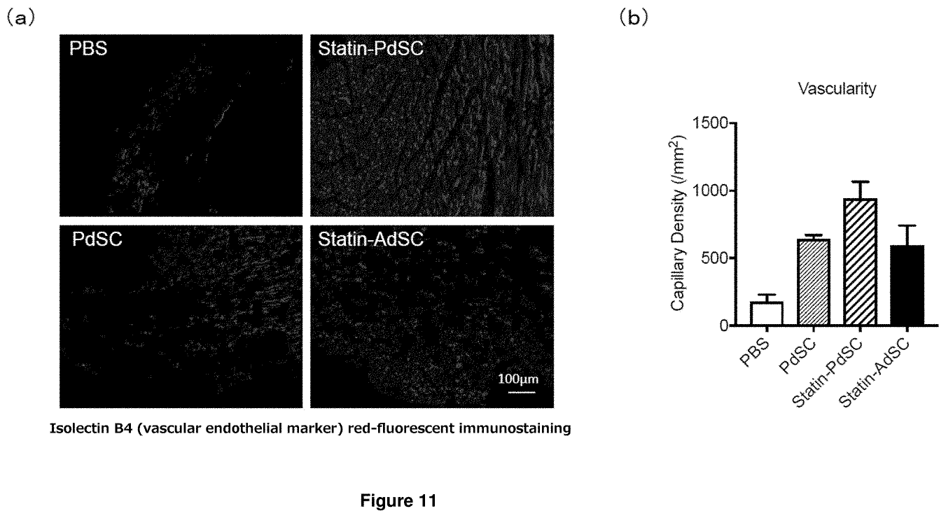

FIG. 11 is a graph showing a result of measuring a blood vessel density of an isolectinsB4-stained segment of an infarct part of a heart of a myocardial infarction model mouse into which PBS, dental pulp-derived stem cells, adipose-derived stem cells comprising statin-encapsulated nanoparticles, or dental pulp stem cells comprising statin-encapsulated nanoparticles was administered; and (B) is a graph showing the measured blood vessel density.

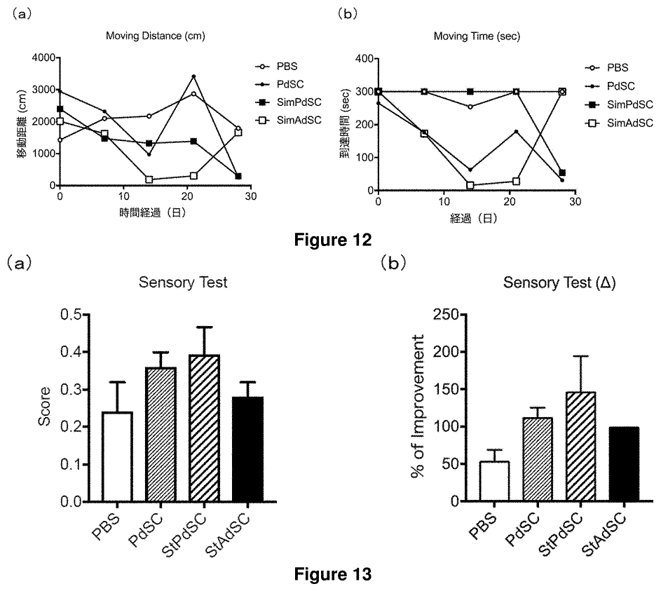

FIG. 12 is a result of storage analysis of a dementia model mouse into which PBS, dental pulp-derived stem cells, adipose-derived stem cells comprising statin-encapsulated nanoparticles, or dental pulp stem cells comprising statin-encapsulated nanoparticles was administered. (a) is a graph showing the moving distance of the mouse until it enters the escape cage by finding the target hole in a Barnes maze test, and (b) is a graph showing the time until the mouse enters the escape cage.

FIG. 13 shows a result of Von Frey test on a modified arthropathy model mouse into which PBS, dental pulp-derived stem cells, adipose-derived stem cells comprising statin-encapsulated nanoparticles, or dental pulp stem cells comprising statin-encapsulated nanoparticles was administered;

(A) shows a score of a threshold that causes an escape reaction after two weeks of administration; and (B) shows the ratio of the score after two weeks of administration to the score on the day of administration.

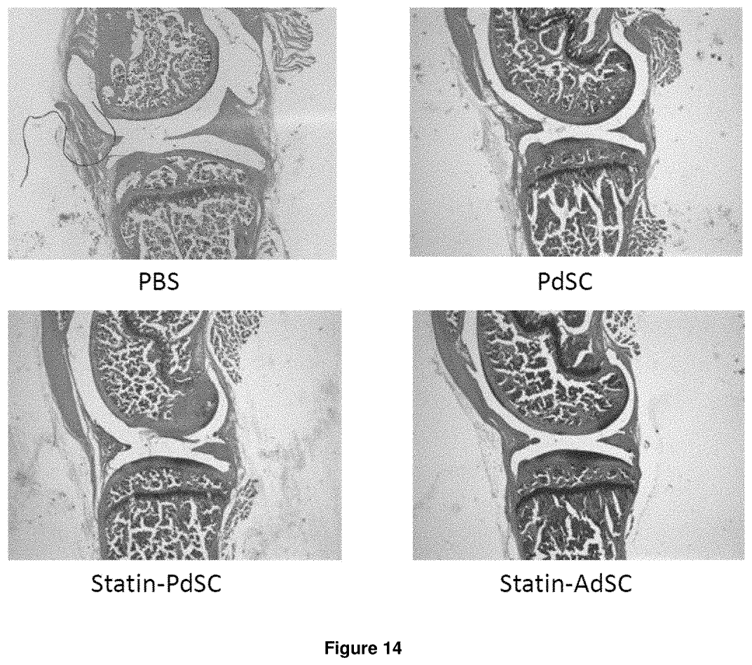

FIG. 14 is a photograph showing a histological analysis result of articular cartilage tissue of an osteoarthritis model mouse into which PBS, dental pulp-derived stem cells, adipose-derived stem cells comprising statin-encapsulated nanoparticles, or dental pulp stem cells comprising statin-encapsulated nanoparticles was administered.

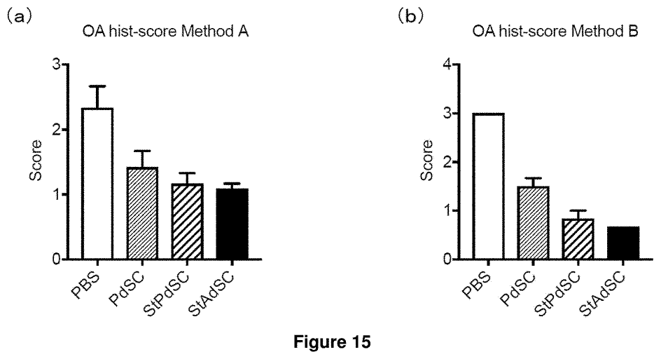

FIGS. 15 (a) and (b) are graphs showing the result of the scoring of the joint damage degree of an osteoarthritis model mouse into which PBS, dental pulp-derived stem cells, adipose-derived stem cells comprising statin-encapsulated nanoparticles, or dental pulp stem cells comprising statin-encapsulated nanoparticles was administered.

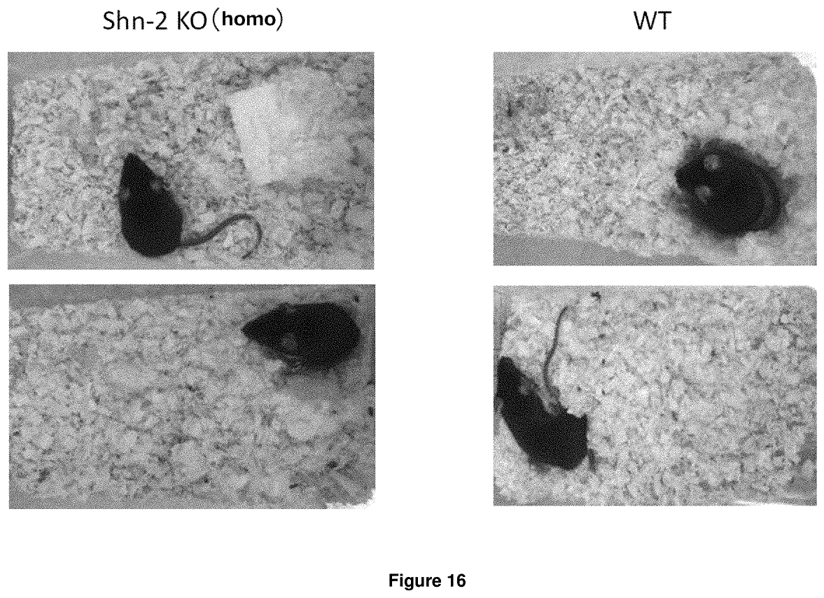

FIG. 16 is a photograph showing the results of the nest making action analysis of a normal mouse and a Shn-2 KO mouse.

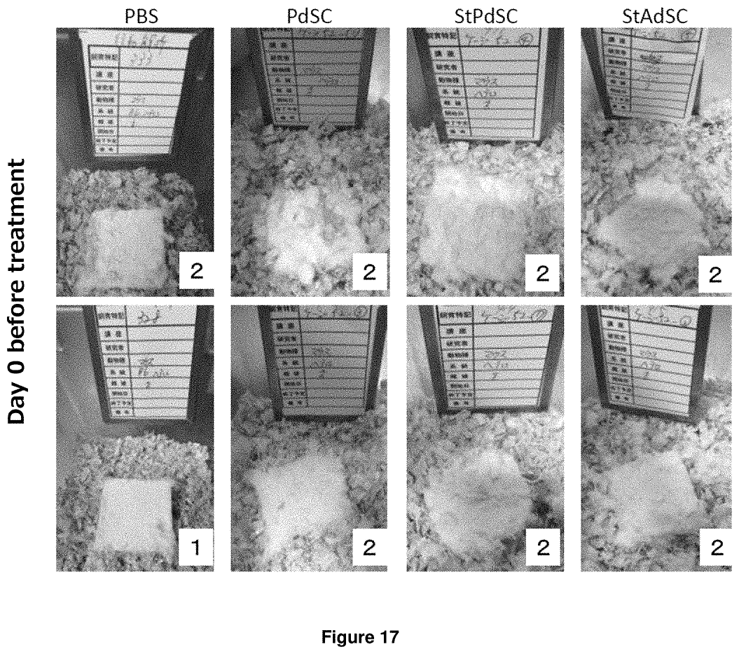

FIG. 17 is a photograph showing the results of the nest making action analysis of Shn-2 KO mice before administration of dental pulp-derived stem cells, adipose-derived stem cells comprising statin-encapsulated nanoparticles, or dental pulp stem cells comprising statin-encapsulated nanoparticles (Day 0).

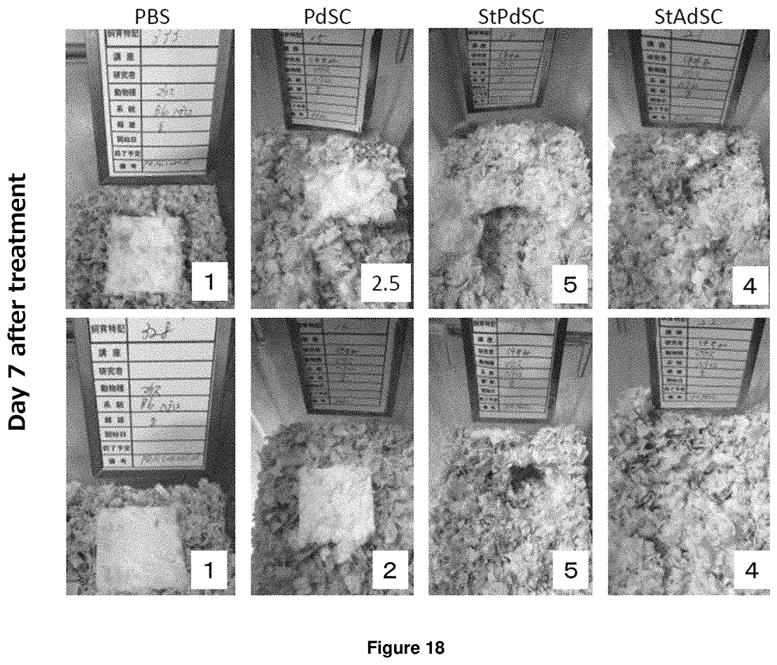

FIG. 18 is a photograph showing the results of the nest making action analysis of SHN-2 KO mice one week after administration of dental pulp-derived stem cells, adipose-derived stem cells comprising statin-encapsulated nanoparticles, or dental pulp stem cells comprising statin-encapsulated nanoparticles (Day 7).

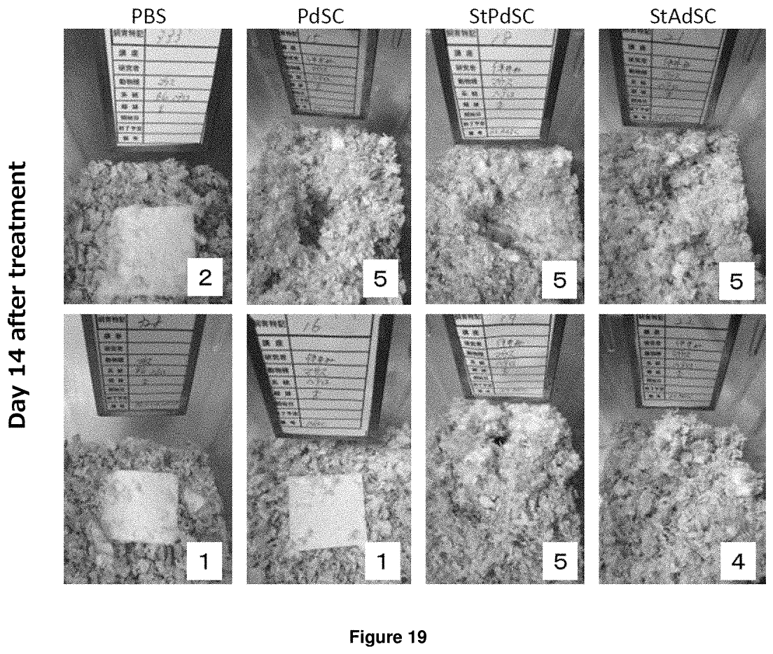

FIG. 19 is a photograph showing the results of the nest making action analysis of SHN-2 KO mice two weeks after administration of dental pulp-derived stem cells, adipose-derived stem cells comprising statin-encapsulated nanoparticles, or dental pulp stem cells comprising statin-encapsulated nanoparticles (Day 14).

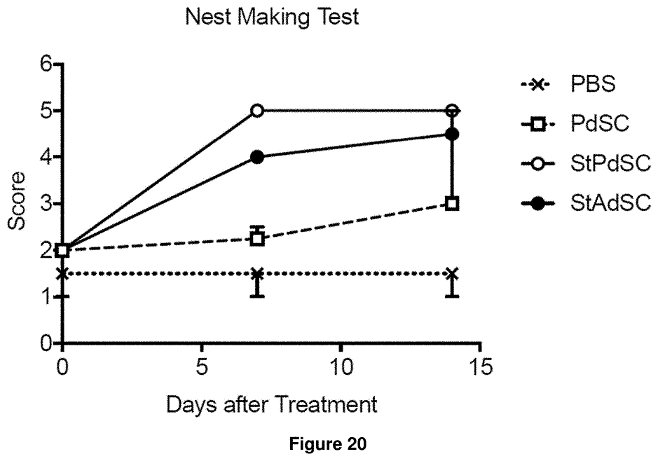

FIG. 20 is a graph reflecting the results of FIGS. 17-19.

Embodiments for carrying out the present invention are described below with reference to the drawings. The following descriptions of the preferred embodiments are described for illustrative purposes, and are not intended to limit the invention, its application method or use thereof.

The statin-encapsulated nanoparticles used in statin-encapsulated nanoparticle preparation of the invention are statin-encapsulated nanoparticles in which statin is encapsulated in nanoparticles comprising polylactic acid glycolic acid copolymer, and used for enhancing a function of dental pulp-derived stem cells. The statin-encapsulated nanoparticle preparation may contain an additive commonly used for formulation such as a stabilizer, a preservative, a buffer, a pH adjuster, an excipient, and like in addition to the statin-encapsulated nanoparticles.

In the present invention, statin comprises all of a compound that is an HMG-CoA (3-hydroxy-3-methylglutaryl-coenzyme A) reductase inhibitor, which includes, for example, simvastatin, lovastatin, pitavastatin, atorvastatin, cerivastatin, fluvastatin, pravastatin, lovastatin, and mevastatin. It is known that statin has a cholesterol lowering action as described above, and large-scale clinical trials revealed that statin can reduce the occurrence of cardiovascular events and the risk of its progression. In addition, many reports have been made on an angiogenesis-promoting effect through vascular endothelial cells and vascular endothelial progenitor cells derived from bone marrow. It is also known to show an anti-inflammatory effect.

In the present invention, the nanoparticle is not limited as long as being a bioabsorbable polymer capable of encapsulating statin. It is preferable to use nanoparticles containing a polylactic acid polymer (polylactic acid: PLA) or a polylactic acid glycolic acid copolymer (PLGA). Since the PLA is hydrolyzed in the body and decomposed into lactic acid and the PLGA is hydrolyzed in the body, decomposed into lactic acid and glycol, and finally becomes water and carbon dioxide, they are harmless and preferable for the body. The weight-average molecular weight of PLA or PLGA used in producing nanoparticles is not limited to the following but can be used, for example, in the range of 5,000-50,000. Even when a material other than PLA and PLGA is used, a person skilled in the art can select a suitable molecular weight material.

In the present invention, the statin-encapsulated nanoparticles can be produced by any method which can be processed to be less than 1000 nm, preferably from about 100 nm to 400 nm, and more preferably from 200 nm to 400 nm when they are measured by a light scattering method from the viewpoint of the uptake efficiency of dental pulp-derived stem cells. It is preferable to produce them by using a spherical crystallization method. Spherical crystallization method is well known as a method capable of designing spherical crystal particles and directly controlling and processing the physical properties by controlling a generation/growth process of crystal in final process of compound synthesis. One of the spherical crystallization methods is a well-known emulsion solvent diffusion method (ESD method).

The emulsion solvent diffusion method is performed by using two kinds of organic solvents including a good solvent capable of dissolving a bioabsorbable polymer such as PLA or PLGA for encapsulating the statin and a poor solvent in which the polymer is not dissolved. First, a polymer such as PLA or PLGA is dissolved in a good solvent, and the statin solution is added to the good solvent and mixed so as not to precipitate the polymer.

When the mixed liquid is dropped into a poor solvent being stirred, since a good solvent rapidly interdiffuses to a poor solvent and the poor solvent rapidly interdiffuses to a good solvent, the interface between the organic solvent phase and the water phase is disturbed, the good solvent is self-emulsified, and an emulsion droplet of submicron size is formed. Thereafter, the mutual diffusion of the good solvent and the poor solvent is further advanced, the solubility of the polymer and the statin such as PLA or PLGA in the emulsion drop is reduced, and as a result, polymer nanoparticles of spherical crystal particles containing statin are generated.

The bioabsorbable polymer and statin used in the production of statin-encapsulated nanoparticles are not limited as long as the obtained statin-encapsulated nanoparticles can enhance the function of dental pulp-derived stem cells, but they are preferably mixed at a rate of 5%, for example. As shown in the example below, when statin-encapsulated nanoparticles are prepared by using 50 mg of PLGA (weight average molecular weight 20000), simvastatin (2.5 mg), acetone (2 mL) and ethanol (0.5 mL) as a good solvent, and 2 wt % PVA solution (10 mL) as a poor solvent, about 50 μg of statin can be encapsulated in 1 mg of nanoparticles. Although the statin-encapsulated nanoparticles are not limited as long as the function of dental pulp-derived stem cells can be enhanced, it is preferable that statin is encapsulated at about 30-60 μg in 1 mg of the nanoparticles.

The dental pulp-derived stem cells which can be used in the present invention are contained in a dental pulp cell population recovered from a dental pulp tissue. As the dental pulp-derived stem cell used in the present invention, the dental pulp cell population containing the pulp-derived stem cells can be used as it is, which is obtainable by recovering a dental pulp cell population from the dental pulp tissue and culturing it with using a medium such as αMEM as needed. The dental pulp cell population containing dental pulp-derived stem cells may be cryopreserved and thawed after cryopreservation. As a preferred embodiment, dental pulp-derived stem cells can be further selected from a cell population containing the dental pulp-derived stem cells, and a cell population composed only of recovered dental pulp-derived stem cells can be used. The method for selecting dental pulp-derived stem cells from dental pulp cells is well known (for example, Yamaza et al. “Immunity property of stem cells from human exfoliated deciduous teeth”. Stem Cell Res Ther. (2010) 1:5)

The “dental pulp tissue” can be collected from any of a deciduous tooth and a permanent tooth, and can be obtained from a dental pulp of an extraction tooth such as a deciduous tooth or a wisdom tooth which has been treated as a medical waste. That is, the dental pulp tissue can be taken out from a tooth that is dental-treated in a dental medical facility, and may be extracted from a natural pull-out tooth or a naturally dropped tooth. The method for taking out the dental pulp tissue from the tooth is well known, and a person skilled in the art can perform it appropriately. When freezing process cannot be carried out immediately on a site, for example teeth that have been dental-treated, the teeth may be preserved and transported by immersing in a medium such as Alpha-Minimum Essential Medium (Alpha-MEM) and storing at a low temperature (eg, 4° C.). The dental pulp can be derived from humans and other mammals (eg, mice, rats, rabbits, dogs, cats, monkeys, sheep, cows, horses). Preferably, the dental pulp-derived stem cell is one derived from a human-derived dental pulp tissue.

The treatment of the statin-encapsulated nanoparticles into the dental pulp-derived stem cells is carried out, for example, by adding the nanoparticles to the culture medium in which the stem cells are cultured. Thus, since the stem cells take up statin-encapsulated nanoparticles by fagocytosis, it is possible to easily incorporate statin-encapsulated nanoparticles into a stem cell without using a special reagent or the like (see, for example, U.S. Pat. No. 6,110,578). The condition of the treatment of the statin-encapsulated nanoparticle to the dental pulp-derived stem cell is not limited as long as the statin-encapsulated nanoparticle is taken into the dental pulp-derived stem cell and the function of the dental pulp-derived stem cell is enhanced. The condition is, for example, preferably 30 minutes to 1 hour at 37° C. In the case of using statin-encapsulated nanoparticles in which about 30-60 μg of statin is encapsulated in 1 mg of nanoparticles, the concentration of nanoparticles to be used in the treatment of a cell population containing dental pulp-derived stem cells is not limited as long as the function of the dental pulp-derived stem cells is enhanced, but is, for example, preferably at a concentration from about 25 μg/5×10⁴ cells to about 200 μg/5×10⁴ cells, more preferably at a concentration from 50 μg/5×10⁴ cells to about 200 μg/5×10⁴ cells.

In one embodiment, the statin-encapsulated nanoparticle according to the present invention enhances the expression of HGF of dental pulp-derived stem cells. HGF gene is a gene involved in angiogenesis and hepatic cell proliferation ability. Particularly, in angiogenesis, HGF shows superior migration ability to smooth muscle cells, compared to VEGF and bFGF which are other angiogenic factors. In addition, angiogenesis by HGF has been reported to have no edema formed by EGF-induced angiogenesis and no inflammation as seen in angiogenesis by bFGF, (T. Kaga et al., “Hepatocyte growth factor stimulated angiogenesis without inflammation: Differential actions between hepatocyte growth factor, vascular endothelial growth factor and basic fibroblast growth factor” Vascular Pharmacology, (2012) Volume 57, Issue 1, 19 August, Pages 3-9). Thus, it suggests that dental pulp-derived stem cells in which the expression of HGF is enhanced by the statin nanoparticles according to the present invention can contribute to the formation of a more normal mature blood vessel. The statin-encapsulated nanoparticle according to the present invention preferably can enhance the expression amount of HGF gene about 1.2 times or more, more preferably about 1.5 times or more, and more preferably about 1.7 times or more. Thus, the dental pulp-derived stem cell having enhanced expression of HGF gene is preferable for treatment of ischemic disease (ischemic heart disease, obstructive arteriosclerosis, thrombosis arterial flame, etc.), cirrhosis or the like.

The dental pulp-derived stem cell treated with the statin-containing nanoparticle according to the present invention, in particular, has an enhanced migration ability and am enhanced angiogenesis factor production ability, and in particular, has enhanced therapeutic effects for various diseases. The function-enhanced stem cell according to the present invention exhibits remarkable effects with a small dose even by intravenous administration or intra-arterial administration without local administration.

In the present invention, the ischemic disease refers to a state in which tissue ischemia is sustained due to stenosis or occlusion of an artery, or a disease caused by ischemia. The ischemic diseases are not limited to the following, but are, for example, ischemic heart diseases such as angina and myocardial infarction, cerebral ischemic diseases such as cerebral infarction, chronic cerebral ischemic diseases such as moyamoya disease, spinal cord deficiency, Ischemic enteropathy such as ischemic colitis and mesenteric artery occlusion, lower limb ischemic diseases such as arteriosclerosis obliterans and Buerger's disease, retinal ischemic diseases such as diabetic retinopathy and like.

In particular, when the dental pulp-derived stem cells treated with the statin-encapsulated nanoparticles according to the present invention are administered intravenously to a patient of ischemic heart disease, the pulp-derived stem cells reach the heart by blood flow and its migration ability, and accumulate and proliferate in the ischemic injury myocardial portion and differentiate into cardiovascular cells. In addition, the accumulated statin-encapsulated nanoparticle-containing dental pulp-derived stem cells promote the production and release of angiogenic factors and promote the regeneration of myocardial tissue by a number of angiogenic factors. In one embodiment, the dental pulp-derived stem cells treated with the statin-encapsulated nanoparticles according to the present invention may be administered intraarterially. When administered intraarterially, the dental pulp-derived stem cells reach the organ which has inflammation, such as the intestine, by the blood flow, accumulate and proliferate in the inflammatory part, produce an anti-inflammatory cytokine, and suppress the activity of the inflammatory cell. As a result, they show a remarkable therapeutic effect on inflammatory diseases.

In the present invention, an inflammatory disease refers to a disease having inflammation as one of its etiologies, and it is not limited to diseases having inflammation as a characteristic symptom such as intestinal flame and pneumonia, and includes diseases in which inflammation is involved in development process thereof such as pulmonary hypertension and dementia. Specifically, the inflammatory disease in the present invention is systemic lupus erythematosus, sceroderma, atopic dermatitis, rheumatoid arthritis, interstitial pneumonia, bronchial asthma, pulmonary hypertension, Inflammatory bowel disease (IBD) such as ulcerative colitis and Crohn's disease, Nerve injury, spinal cord injury, stroke (sequelae after cerebral infarction and cerebral hemorrhage), muscular atrophic lateral sclerosis, chronic inflammatory demyelinating polyneuritis, schizophrenia, dementia, rejection during organ transplantation, chronic nephritis (Nephrosclerosis), and like.

In the present invention, a neurodegenerative disease refers to a disease causing a specific nerve cell group of nerve cells in a central nervous system (for example, brain or spinal cord) to gradually fall off due to a disorder, lowering exercise capacity, lowering balance feeling, lowering of muscular strength, and/or lowering cognitive ability. The neurodegenerative diseases include, but are not limited to, amyotrophic lateral sclerosis (ALS), Parkinson's syndrome (Parkinson's disease, etc.), Alzheimer's disease, Lewy-type dementia, cortical basal nucleus degeneration, progressive nuclear paralysis (PSP), Huntington's disease, multi-system atrophy (MSA) (Black streak degeneration (SND), Shy-Drager syndrome (Shy-Drager syndrome), Olivopontocerebellar atrophy (OPCA), etc.), Spinocerebellar degeneration (SCD) (Spinocerebellar imbalance (SCA3, commonly known Machado-Joseph's disease, etc.), Friedreich's ataxia (Friedreich's ataxia, etc.), and the like.

As described above, since the dental pulp-derived stem cells can gradually release the encapsulated statin by hydrolyzing the statin-encapsulated nanoparticles incorporated in the cells, when the dental pulp-derived stem cells are administered into the body, the dental pulp-derived stem cells can gradually release the statin after administration, and can provide a further anti-inflammatory effect by the released statin.

In one embodiment, a cell preparation containing the dental pulp-derived stem cells treated with statin-encapsulated nanoparticles according to the present invention can be used as a cell preparation for improving pain. In one embodiment, the cell preparation containing the dental pulp-derived stem cells treated with the statin-encapsulated nanoparticles according to the present invention can be used as a cell preparation for improving cartilage damage.

The dental pulp-derived stem cell treated with the statin-encapsulated nanoparticle has a therapeutic effect of dementia for a knock-in mouse in which a gene mutation is inserted into the amyloid beta region of mouse APP gene as a dementia model mouse. It has been reported that a knock-in mouse into which a gene mutation is inserted into the amyloid beta region of mouse APP gene increases the production ratio of toxic amyloid beta species (AP 42) in the brain, resulting in the promotion of the formation of amyloidosis, and that nerve inflammation and the loss of synapse is recognized. Accordingly, one embodiment of a cell preparation containing the dental pulp-derived stem cells treated with statin-encapsulated nanoparticles according to the present invention can be used as a preparation for the treatment of neuroinflammation caused by the accumulation of amyloid beta.

The dental pulp-derived stem cells treated with the statin-encapsulated nanoparticles according to the present invention have the therapeutic effect of schizophrenia for SHN-2 knockout (KO) mice that are schizophrenia model mice. Accordingly, one embodiment of a cell preparation containing the dental pulp-derived stem cells treated with statin-encapsulated nanoparticles according to the present invention can be used as a therapeutic preparation for schizophrenia caused by the expression reduction or knockout of SHN-2. Further, in the brain of the SHN-2 knockout (KO) mouse, it has been reported that neuroinflammation is caused by activation of astroglia cells, and the dental pulp-derived stem cells treated with the statin-encapsulated nanoparticles according to the present invention are considered to be capable of suppressing neuroinflammation caused by activation of astroglia cells.

### EXAMPLE

Examples for explaining in detail a statin-encapsulated nanoparticle preparation for enhancing the function of a stem cell according to the present invention and the function-enhanced stem cell containing the same are shown below.

First, a method for producing statin-encapsulated nanoparticle is described. In the examples, simvastatin is used as a statin, and polylactic acid/a glycolic acid copolymer (PLGA) is used as the nanoparticle.

50 mg of PLGA (weight average molecular weight 20000) and 2.5 mg of simvastatin were dissolved in a mixture of 2 mL of acetone and 0.5 mL of ethanol to prepare a polymer solution. The polymer solution was dropped into 10 ml of 2 wt. % PVA solution stirring at room temperature and 500 rpm to obtain a simvastatin-encapsulated PLGA nanoparticle suspension. Subsequently, the organic solvent (acetone, ethanol) was distilled away while stirring at room temperature and 500 rpm. After solvent distillation for about 5 hours, the suspension was centrifuged at 4° C. and 6000 g for 30 minutes, and the precipitate was recovered and resuspended in distilled water. The operation of the centrifugation and the resuspension to distilled water was carried out three times. Thereafter, the suspension was freeze-dried overnight, and simvastatin-encapsulated PLGA nanoparticles were obtained, which were used in the following test as statin-encapsulated nanoparticles.

The dental pulp-derived stem cells used in this example were prepared as follows. The dental pulp tissue was taken out by using tweezers or the like from an extracted deciduous tooth collected from patient (4-13 year) in an affiliated dental clinic. The extracted dental pulp tissue was finely cut by using scalpel or the like, and cell dispersion was performed by using an enzyme solution for tissue dispersion such as Accutase. The dispersed cells were suspended in a serum-containing alpha-MEM medium to count the number of cells. The cells were cultured at 37° C. and 5% CO₂ with a cell culture flask having an appropriate size based on the results of counting the number of cells. A total amount of culture medium was exchanged every 2-3 days. The primary cultured cells were cultured in a flask until a semi-confluent state (cell density: 70-80%).

The semi-confluent state was confirmed by microscopic visualization, and the whole amount of the medium was removed. In order to completely remove the medium containing serum, D-PBS (−) was used to wash the bottom surface of the flask, and this operation was repeated twice. After washing, a proper amount of TrypLE Select (Gibco) was added, spread over the entire bottom surface of the flask, and incubated at 37° C. At a timing when a part of the cells were peeled from the bottom surface of the flask, the cells were separated from the bottom surface of the flask by slowly circulating the solution in the flask. The flask was tilted and serum-containing α-MEM medium was added so as to flow through the entire flask, and the cells were peeled off to prepare a cell suspension. The cell suspension was recovered to a 15 mL tube. A new alpha-MEM medium was added to flow through the entire flask, and the cells were peeled off. The cell suspension was recovered to the 15 mL tube. The recovered cell suspension was centrifuged, and the supernatant was removed, and the serum-containing alpha-MEM medium was added and suspended to count the number of cells. The cells were cultured at 37° C. and 5% CO₂ using a cell culture flask having appropriate size to the cell density of 2,000 to 5,000 cells/cm². The enlargement culture was repeated for two passages.

After confirming that the cells in the enlarged culture are in a semi-confluent state (cell density: 70-80%), the culture supernatant was recovered into a new 50 ml tube. The cells in the flask after the supernatant recovery were frozen, and the culture supernatant collected in the 50 ml tube was used for the following examinations. The safety of the cell was confirmed by an examination including HBV quantitative determination, HCV quantitative determination, HIV quantitative determination, parvovirus IgG, parvovirus IgM, CMV-IgG, CMV-IgM, FTA-ABS, STD-Mycoplasma identification, and endotoxin. Washing of the bottom surface of the flask was performed by using D-PBS (−) in order to completely remove the culture medium component containing serum from the inside of the flask after confirming the semi-confluent state in the enlarged culture and removing the culture supernatant, and the operation was repeated twice on the respective flasks. The TrypLE select was added to each flask, spread over the entire bottom surface of the flask, and the flasks were incubated at 37° C. At a timing when a part of the cells was peeled from the bottom surface of the flask, the cells were separated from the bottom surface of the flask by slowly circulating the solution in the flask. The alpha-MEM medium was added to flow through the entire flask, and the cells were separated to prepare a cell suspension. The cell suspension was recovered to a 15 ml tube. 3 ml of alpha-MEM medium was added to flow throughout each flask, the remaining cells in the flask were peeled off to prepare a cell suspension, and the cell suspension was recovered to a 15 ml tube. The recovered cell suspension was centrifuged, its supernatant was removed, and D-PBS (−) was added and suspended to count the number of cells. The supernatant was removed again by centrifugation. Cellbanker 2 which is a cryopreservation solution in an amount according to the number of cells (final concentration: 0.9 to 1.3×10⁶ cells/ml) was added and gently pipetted. 1 ml of the cryopreservation solution containing cells was transferred to a cryotube one by one. The cryotube was placed in Bicel, frozen at −80° C. freezer, and transferred to a liquid nitrogen tank within 3 days.

About 70% of the cryopreservation solution containing cryopreserved dental pulp stem cells was rapidly thawed in constant temperature tank. About 70% thawed cryopreservation solution was added to an alpha MEM medium heated to 37° C. After thawing, centrifugation was performed, and the supernatant was removed. A new medium was added to count the number of cells. The culture was carried out in an incubator of 37° C. and 5% CO₂ using a cell culture flask having appropriate size to a cell density of 2,000 to 5,000 cells/cm². After seeding, the whole amount of the medium was exchanged every 2-3 days. The expanded culture was repeated until the necessary number of cells to be used for the test was obtained, and a cell population containing the dental pulp-derived stem cells was prepared. Such prepared cell population which contains the dental pulp-derived stem cells was used in the following examples. In the following, a cell population containing the dental pulp-derived stem cells is conveniently referred to as “dental pulp-derived stem cell”.

Next, the following tests were performed to examine an enhancement of the function, such as a migration ability and an ability to produce angiogenesis factors, of dental pulp-derived stem cells by the simvastatin-encapsulated nanoparticles.

First, the migration ability of dental pulp-derived stem cells (PdSC) was examined using a migration test kit (Transwell®). Concretely, a cell strain of dental pulp-derived stem cell (#221) was dispensed into an alpha-MEM so as to be at 5×10⁴ cells/500 μl/tube in a plurality of microtubes; and simvastatin-encapsulated PLGA nanoparticles were added so as to be in an amount of 0 μg/5×10⁴ cells, 25 μg/5×10⁴ cells, 50 μg/5×10⁴ cells, 100 μg/5×10⁴ cells, or 200 μg/5×10⁴ cells and left to stand for 18 hours. The dental pulp-derived stem cells in 20% FBS αMEM medium were seeded at 5×10⁴ cells on the porous membrane of each well of Transwell plate, and after 6 hours, the number of cells passed through the membrane of Transwell plate was measured. As a control, the dental pulp-derived cells seeded at 5×10⁴ cells in 0% FBS αMEM medium without treating simvastatin-encapsulated PLGA nanoparticles was used. The result was shown in FIG. 1.

As shown in FIG. 1, when treated with low-concentration simvastatin-encapsulated PLGA nanoparticles of 25 or 50 μg/5×10⁴ cells, there was no change in the migration ability of the denal pulp-derived stem cells compared to those that were not treated, but when treated with high-concentration simvastatin-encapsulated PLGA nanoparticles of 100 or 200 μg/5×10⁴ cells, particularly 200 μg/5×10⁴ cells, the migration ability of the dental pulp-derived stem cells was increased. As a result, it has been shown that a relatively high concentration of simvastatin-encapsulated PLGA nanoparticles can enhance the migration ability of dental pulp-derived stem cells.

Next, the analysis of the mRNA expression amount of an angiogenic factor in the dental pulp-derived stem cell was performed by a quantitative PCR method in order to examine the effect of the simvastatin-encapsulated nanoparticles on the angiogenic factor production ability of the dental pulp-derived stem cells. In this example, the expression amount of intracellular mRNA of HGF was measured as the angiogenic factor.

First, three cell strains of the dental pulp-derived stem cells (#221) were dispensed to microtubes so as to be at 1×10⁵ cells/1 ml/tube, and simvastatin-encapsulated PLGA nanoparticles were added to the microtubes so as to be at a concentration of 0 μg/mL, 50 μg/mL, 100 μg/mL, 200 μg/mL, or 400 μg/mL, and the microtubes were left to stand for 30 minutes. Thereafter, the cells were washed with PBS and seeded in a 6 well culture dish using 20% FBS αMEM medium. The cells were recovered after 48 hours, and their RNA was extracted by using a NucleoSpinRNA kit (Takara Bio). Then, mRNA expression amount of HGF in each cell was measured by a quantitative PCR method using primers relating to the DNA sequence of HGF. The measurement was performed by synthesizing cDNA from the extracted RNA by using an RverTra Ace qPCR RT kit (TOYOBO) and by reacting the cDNA with SsoFastEvaGreen Mastermix agent (Biorad) and primers in a thermal cycler (Cfxconnect Biorad) (one cycle at 95° C. for 30 seconds and 40 cycles at 95° C. for 5 seconds/at 56° C. for 5 seconds). For comparison, three cell strains (#101, #103, #104) of adipose-derived stem cells (AdSC) were treated in the same way as described above to measure mRNA expression amount of HGF. The measurement result is shown in FIG. 2. The result indicates each ratio of the expression amount of the above factor based on the mRNA expression amount of GAPDH.

As shown in FIG. 2, the increase of HGF expression was observed in any strain of the dental pulp-derived stem cells treated with especially the low-concentration simvastatin-encapsulated PLGA nanoparticles of 50 μg/mL (25 μg/5×10⁴), 100 μg/mL (50 μg/5×10⁴). On the other hand, although the increase of HGF expression was observed in one strai of the adipose-derived stem cells treated with simvastatin-encapsulated PLGA nanoparticles, there was no increase of HGF expression in two strains.

As described above, it has been found that statin-encapsulated nanoparticles can enhance the function of dental pulp-derived stem cells as a result of examining the enhancement of functions such as the migration ability and the ability to produce angiogenesis factors of dental pulp-derived stem cells due to statin-encapsulated nanoparticles. By enhancing these functions, dental pulp-derived stem cells are considered to be advantageous for the treatment of various diseases.

Next, a myocardial infarction treatment effect of the dental pulp-derived stem cells containing statin-encapsulated nanoparticles according to the present invention was examined using a myocardial infarction model mouse.

First, ischemia induction (anterior descending coronary artery ligation model) was conducted to 10-12 weeks old male BALB/c nude mice (Day 0), and 3 days later (Day 3), PBS (phosphate buffered saline), dental pulp-derived stem cells (5×10⁴ cells), and dental pulp-derived stem cells containing statin-encapsulated PLGA nanoparticles (100 μg/5×10⁴ cells, #221 strain) or adipose-derived stem cells containing statin-encapsulated PLGA nanoparticles (100 μg/5×10⁴ cells, #104 strain) was administered to the tail vein. After 53 days (Day 56), cardiac ultrasound image diagnosis was performed, and the heart tissue analysis was then performed by dissecting the heart. In cardiac ultrasonic image diagnosis, a left ventricular inner fractional shortening (FS) and a left ventricular end-diastolic diameter (LVDd) were measured, and the rate of change from Day 3 to Day 56 was evaluated. As the histological analysis, the section of the infarct portion in the taken-out heart of each group was prepared by a conventional method, fibrosis area ratio, fibrosis length ratio, infarcted myocardial wall thickness ratio of the infarct portion were analyzed by using the Masson trichrome stain. A photograph of the heart at Day 56 is shown in FIGS. 3 and 4, and photographs of Masson trichrome-stained section (tissue specimen) at different portions are shown in FIGS. 5-8, and the results of the left ventricular myocardial morphological analysis using the tissue samples are shown in FIG. 9, and the results of the cardiac ultrasound image diagnosis are shown in FIG. 10.

As shown in FIGS. 3 and 4, compared with PBS group and PdSC group, which dental pulp-derived stem cells were administered to, as control, it was confirmed from the appearance that the expansion of the heart was suppressed in the group to which the adipose-derived stem cells containing the statin-encapsulated nanoparticles was administered (statin-AdSC group), and it was also confirmed from the appearance that the expansion of the heart was further suppressed in the group to which the dental pulp-derived stem cells containing the statin-encapsulated nanoparticles was administered (statin-PdSC group). Although many mice died in PBS group and PdSC group, only one of the 5 mice died in statin-AdSC group, and there is no mouse dead in the statin-PdSC group.

As described above, FIGS. 5-8 shows photographs of Masson trichrome-stained section at different portions of heart on Day 56. The myocardial cell portion which is necrosed by ischemia become fibrotic by replacing with fibroblasts. The area of the fiberized and blue dyed scar part is indicated by an arrow. As shown in FIGS. 5-8, a wide area was fiberized in PBS group as a control; on the other hand, the fibrous area was shrinking in PdSC group in which dental pulp-derived stem cells had been administered and the group to which adipose-derived stem cells containing statin-encapsulated nanoparticles had been administered (statin-AdSC group); and the fiberized regions were hardly seen in the group to which dental pulp-derived stem cells containing statin-encapsulated nanoparticles had been administered.

FIG. 9 shows the results of analysis on the basis of FIGS. 5-8, and concretely, FIG. 9(a) shows a ratio of the fiberized portion turned blue in each group when the whole area of the heart is 100%, and FIG. 9 (b) shows a ratio of the length of the fiberized portion turned blue in each group when the length of the whole circumference is 100%, and FIG. 9(c) shows a ratio of the thickness of the myocardial wall which fiberized and turned blue when the thickness of the normal myocardial wall is 100%. As shown in FIGS. 9(a) and (b), many regions became fibrotic in PBS group as control, an improvement was observed in PdSC group and the statin-AdSC group, and further improvement was observed in the statin-PdSC group. As shown in FIG. 9 (c), in the same manner, the thickness of the myocardial wall in PBS group as control was extremely thin, and an slight improvement was observed in PdSC group and the statin-AdSC group, and remarkable improvement was observed in statin-PdSC group.

Next, the results of the cardiac ultrasonic image diagnosis are explained with reference to FIG. 10. FIG. 10(a) shows a change rate (ΔLVDD) of the left ventricular end-diastolic diameter (LVDD) at Day 56 in each group to the left ventricular end-diastolic diameter at Day 3, and FIG. 10(b) shows a change rate (ΔFS) of the left ventricular inner fractional shortening (FS) at Day 56 in each group to the left ventricular inner fractional shortening at Day 3. As shown in FIG. 10(a), remarkable hypertrophy was observed in PBS group, and the suppression effect of such hypertrophy was high in the order of statin-PdSC group, statin-AdSC group, and PdSC group. As shown in FIG. 10(b), the left ventricular inner fractional shortening (FS) was deteriorated in PBS group, and no improvement was observed in PdSC group. On the other hand, an improvement was observed in statin-AdSC group, and a further improvement was observed in statin-PdSC group over statin-AdSC group.

As a result, it is possible to improve the cardiac function by using only dentalpulp-derived stem cells but also to obtain a more remarkable improvement effect by using dental pulp-derived stem cells incorporating the statin-encapsulated nanoparticles. The improvement effect was superior to the case of using adipose-derived stem cells incorporating statin-encapsulated nanoparticles.

In order to examine the blood vessel density in the ischemic boundary region, fluorescence staining was performed by using an antibody which binds to an isolectin B4 which is a plant-derived protein that binds to a glycoprotein expressed in vascular endothelial cells, and the size of the dyeing region in the microscope field was measured. The measurement result was graphed as a blood vessel density. The result is shown in FIG. 11. As shown in FIGS. 11(a) and (b), the blood vessel density was increased in PdSC group and statin-AdSC group as compared with PBS group, and it was confirmed that the blood vessel density was further increased in statin-PdSC group as compared with the other groups. As a result, it is considered that angiogenesis is promoted in the infarct peripheral part (ischemic part) to contribute to myocardial tissue regeneration due to the administration of dental pulp-derived stem cells incorporating statin-encapsulated nanoparticles.

The above results indicate that it is possible to obtain a superior therapeutic effect on ischemic heart diseases such as myocardial infarction according to the dental pulp-derived stem cells containing the statin nanoparticles of the present invention. Specifically, the statin-encapsulated nanoparticle of the present invention can enhance the function of the migration ability and the ability to produce angiogenic factors in the dental pulp stem cells, and the function-enhanced stem cells are integrated and proliferated in the infarct part, and release the angiogenesis factor to promote angiogenesis of the infarct part. Further, by differentiating the accumulated stem cells into the myocardium, regeneration of the myocardium is promoted, and as a result, the superior therapeutic effect of ischemic heart disease such as myocardial infarction is obtained. The stem cells are useful for the treatment of ischemic heart disease because they can slowly release the taken-in statin, thereby obtaining various effects peculiar to statin such as angiogenesis effect of statin itself. In particular, compared with the case of using adipose-derived stem cells, when dental pulp-derived stem cells are used, it shows superior effects on (i) suppression of fibrosis, (ii) improvement of the thickness of the myocardial wall, and (iii) promotion of angiogenesis.

Next, dementia model mice were used to examine the therapeutic effect of the dental pulp-derived stem cells containing statin-encapsulated nanoparticles according to the present invention. The methods and results are described below. As a dementia model mouse, C57BL/6-App<tm3(NL-G-F)Tcs> obtained from Riken BioResource Research Center was used. PBS, dental pulp-derived stem cells (#221), adipose-derived stem cells containing statin-encapsulated nanoparticles or dental pulp-derived stem cells (#221) containing statin-encapsulated nanoparticles were slowly administered to the model mice from their tail vein. The dosage of each stem cell was 1×10⁴ cells/mouse and 200 μl of PBS was used as a solvent. As the statin-encapsulated nanoparticles, the simvastatin-encapsulated PLGA nanoparticles were used, and 20 μg of these nanoparticles were cultured with adipose-derived stem cells or dental pulp-derived stem cells at 37° C. for 30 minutes to obtain adipose-derived stem cells or dental pulp-derived stem cells containing statin-encapsulated nanoparticles. A well-known Barnes maze test was performed as a storage analysis for each of the mice to which those have been administered. As the day of administration is defined as Day 0, the time and the moving distance until a mouse finds a target hole provided only in one of 20 circles provided on the peripheral edge part of a bronze maze table and arrives at an escape cage communicating with the target hole were measured as a storage analysis on Day 0, Day 7, Day 14, Day 21, and Day 28, respectively. Storage training was performed one by one on the morning and the afternoon of the previous day of the day conducting the measurement (storage analysis).

Each mouse was rearing in one cage together with a plurality of mice, divided into individual cages one hour or more before the storage training and the storage analysis, and then made into an environment. The storage training first placed the mouse in a white cylindrical container arranged at the center of the maze table for one minute, then removed the cylindrical container from the maze table and sounded an ultrasonic buzzer which the mouse is disliked. After that, the mouse searched a target hole for 3 minutes, and the ultrasonic buzzer was stopped at the time that the mouse entered the escape cage. However, when the mouse did not enter the escape cage even after 3 minutes, the mouse was put in a transparent cylindrical container, and the mouse was forcibly put into the escape cage by taking a time of about 30 seconds while showing the surrounding environment. The mouse acclimatized to the environment for 1 minute after being placed in the escape cage. The storage training was repeated 3 times.

In the storage analysis performed on the next day of the storage training, first, the mouse was allowed to stand for one minute in the white cylindrical container arranged in the center of the maze table, and then the cylindrical container was detached from the maze table, and the ultrasonic buzzer having the frequency that the mouse dislikes was sounded, and action tracking recording was started. Then, the ultrasonic buzzer was stopped at the point of time when the mouse searches the target hole and enters the escape cage, and stops the action tracking recording. In the action tracking record, a moving distance (a target arrival moving distance) and a time (target arrival time) until the mouse enters the escape cage from sounding of the ultrasonic buzzer were measured by using Limelite software (ActiMetrics, Inc. IL, USA), which is a behavior analysis system. The results of storage analysis in each mouse were shown in FIG. 12.

As shown in FIGS. 12(a) and (b), at Day 0, the moving distance and the time until reaching the escape cage were long in each group of PBS-administered dementia model mouse, dental pulp-derived stem cells (PdSC)-administered dementia model mouse, the dementia model mouse in which adipose-derived stem cells containing statin-encapsulated nanoparticles were administered (SimAdSC), and the dementia model mouse in which dental pulp-derived stem cells containing statin-encapsulated nanoparticles were administered (SimPdSC). However, in accordance with the passage of time on Day 7 and Day 14, the moving distance and the time until reaching the escape cage were remarkably shortened especially in SimAdSC group as compared with the PBS group. However, such effect was diminished on day 28 in SimAdSC group. On the other hand, in the SimPdSC group, the moving distance was gradually shortened and the moving distance and the time until reaching the escape cage at the 28th day were remarkably shortened. As a result of this example, it has been suggested that the symptoms of dementia can be improved by dental pulp-derived stem cells containing statin-encapsulated nanoparticles according to the present invention.

Next, a therapeutic effect on osteoarthritis of dental pulp-derived stem cells containing statin-encapsulated nanoparticles according to the present invention was examined by using a osteoarthritis (OA) model mouse. The method and results were described below. As OA model mouse, a well-known model mouse, which is prepared from cutting the anterior cruciate ligament of the right knee joint of BALB/c mouse and resecting the inner meniscus of BALB/c mouse, was used. Concretely, the anterior cruciform ligament in a right knee joint of a BALB/C nude mouse (male, 10 week old) was cut, the inner meniscus was also cut, and in order to induce OA, Treadmill exercise (30 minutes/a day, 15° inclination, 27-33 cm/sec) was applied to the mouse. On Day 21 after surgery, PBS, dental pulp-derived stem cells (PdSC; #221), adipose-derived stem cells containing statin-encapsulated nanoparticles (StAdSC; #221), or dental pulp-derived stem cells containing statin-encapsulated nanoparticles (StPdSC) were topically administered to the right knee joint of the mouse using a 29G injection needle. The dose of PBS was 10 μl. The dosage of the dental pulp-derived stem cells was 1×10⁴ cells/mouse, and 10 μl of PBS was used as solvent. As the statin-encapsulated nanoparticles, the simvastatin-encapsulated PLGA nanoparticles were used, and 20 μg/mL of such nanoparticles and adipose-derived stem cells or dental pulp-derived stem cells (1×10⁴ cells) were co-cultured for 30 minutes to 1 hour to obtain stem cells containing statin-encapsulated nanoparticles.

Also, a well-known von Frey test was performed as a pain test at the day of administration and at day after two weeks from the day of administration. The test measured a threshold of an escape reaction by applying plural filaments that can provide a determined force to the sole of the rat hind limb. The result was shown in FIG. 13. In FIG. 13(a) shows a score (the value indicates the weight (g) required to bend the filament) of the threshold for causing an escape reaction which was measured two weeks after administration, and FIG. 13(b) shows a ratio of the score after two weeks from the administration to the score on the day of administration. As shown in FIG. 13, pain was improved in PdSC group and StAdSC group as compared with the PBS group, and a result of further improvement was obtained in StPdSC group.

In addition to the Von Frey test, after the Von Frey test in two weeks after the above administration, the mouse was euthanized and dissected, and the articular cartilage tissue of the right knee was collected to perform histological analysis. Concretely, the articular cartilage tissue of the right knee in each group was collected, a thin cut sample was prepared after fixing with 4% paraformaldehyde solution, and Safranin O stain was performed. Safranin O was a staining reagent for dyeing a cartilage substrate. FIG. 14 shows the stained photographs of the articular cartilage tissue sections of each group.

As shown in FIG. 14, in the group in which PBS was administered to an OA-induced mouse (PBS group), the cartilage layer of the bone head of the tibia was extremely thin, and staining with Safranin O (dark gray in the figure) was not observed in the cartilage layer. On the other hand, in the group in which dental pulp-derived stem cells were administered to an OA-induced mouse (PdSC group), the cartilage layer of the bone head of the tibia was thick compared with the PBS group, and the staining with Safranin O was slightly observed. Also, in both of the group in which adipose-derived stem cells containing statin-encapsulated nanoparticles were administered to an OA-induced mouse (Statin-AdSC group) and the group in which dental pulp stem cells containing statin-encapsulated nanoparticles were administered to an OA-induced mouse (Statin-PdSC group), the cartilage layer of the bone head of the tibia was further more thick than that of the PdSC group, and a large area stained with Safranin O was also observed.

Further, for each group, the degree of joint damage was scored by histopathological findings. Scoring criteria was based on Osteoarthritis and Cartilage 18 (2010) S17-S23 and Osteoarthritis and Cartilage 13 (2005) 632-642 and evaluated as follows.

TABLE 1

| Score | Histopathological findings in cartilage layer |
|---|---|
| 0 | Normal |
| 0.5 | Decrease of regions dyed with safranine O (tissue construction was |
| | maintained) |
| 1 | Small amount of fibrin deposition (no decrease of cartilage tissues) |
| 2 | Crack (limited in cartilage outer layer + a few amount of decrease |
| | of the surface thin film) |
| 3 | Crack + erosion (reached calcification cartilage layer + equal to |
| | or less than 25% of periphery length) |

TABLE 2

| Score | Cartilage damage |
|---|---|
| 0 | Normal |
| 1 | Cartilage damage in cartilage surface |
| 2 | Cartilage damage in upper tide line |
| 3 | Cartilage defects reaching the calcified cartilage layer |
| 4 | Exposure of subchondral bone |

The results evaluated based on the scale shown in Table 1 are shown in FIG. 15(a), and the results evaluated based on the scale shown in Table 2 are shown in FIG. 15(b). As shown in FIGS. 15(a) and (b), cartilage damage was improved in PdSC group compared to PBS group, and further improvement was observed in StAdSC group and StPdSC group.

As a result, it becomes clear that the symptoms of osteoarthritis can be improved by stem cells containing statin-encapsulated nanoparticles.

Next, a therapeutic effect on schizophrenia of dental pulp stem cells containing statin-encapsulated nanoparticles according to the present invention was examined using a schizophrenia model mouse. The method and results are described below.

C57B6/J mouse whose schnurri-2 (Shn-2) gene had been knocked out (RIKEN BRC) was used as the schizophrenia model mouse. It is known that the brain of Shn-2 knockout (KO) mouse has extremely high similarity to the features reported in the brain of the schizophrenia patient (K Takao et al. Neuropsychophystereology (2013), 38, P1409-1425). Actually, a nest making action of Shn-2 KO mouse was observed to examine whether or not the abnormal action is recognized compared with a normal mouse. A felt was given to the normal mouse (WT) and the Shn-2 KO mouse, and it was observed whether or not the mouse gnaws on the felt to lay it in the nest. The results are shown in FIG. 16.

As shown in FIG. 16, normal mice gnawed all of the felts to lay them, but Shn-2 KO mouse barely gnawed the felts. As a result, the mouse whose Shn-2 is knocked out can be used as a schizophrenia model.

Therefore, the therapeutic effect of schizophrenia of dental pulp stem cells containing statin-encapsulated nanoparticles according to the present invention was examined by using the Shn-2 KO mouse. First, PBS, dental pulp-derived stem cells (PdSC), adipose-derived stem cells containing statin-encapsulated nanoparticles (StAdSC), or dental pulp-derived stem cells containing statin-encapsulated nanoparticles (StPdSC) were administered intravenously to Shn-2 KO mouse. The dose of mouse adipose-derived stem cells was 1×10⁴ cells/mouse. Simvastatin-encapsulated PLGA nanoparticles (50 μg) were used as the statin-encapsulated nanoparticles, and those nanoparticles and adipose-derived stem cells or dental pulp-derived stem cells were cocultured for 30 minutes to 1 hour to obtain stem cells containing statin-encapsulated nanoparticles. A felt was placed in the cage in which each mouse is after administration, and the condition of the felt was observed after one week and after two weeks. Also, as shown in Table 3 below, a score is applied on the basis of the condition of the felt. FIGS. 17 to 20 show observation results and scores.

| Score | Felt condition |
|---|---|
| 1 | Not noticeably touched (90% or more intact) |
| 2 | Partially avoiding (40~50%) |
| 3 | Partially chopped |
| 4 | A flat nest exists |
| 5 | A complete nest exists |

As shown in FIGS. 17 to 20, in PBS group, the nest making behavior was not almost seen even after administration, and symptoms of schizophrenia can be seen strongly. In PdSC group, there was also an individual which took a nest making action two weeks after administration, but some individuals did not show any nest making behavior. On the other hand, in the group in which adipose-derived stem cells containing statin-encapsulated nanoparticles were administered (StAdSC group) or the group in which dental pulp-derived stem cells containing statin-encapsulated nanoparticles were administered (StPdSC), a nest making action was seen after one week of administration, and remarkable recovery of symptoms was observed. From these results, it is suggested that stem cells containing statin-encapsulated nanoparticles can improve symptoms of schizophrenia.

From the above results, statin-encapsulated nanoparticles according to the present invention can enhance the function of dental pulp-derived stem cells, and the function-enhanced stem cells have therapeutic effects on ischemic heart disease, inflammatory diseases, and the like since they are useful for the treatment of those diseases.

1. A statin-encapsulated nanoparticle preparation for enhancing a function of a dental pulp-derived stem cell, the statin-encapsulated nanoparticle preparation comprising a statin-encapsulated nanoparticle in which statin is encapsulated in nanoparticles containing a bioabsorbable polymer.

2. The statin-encapsulated nanoparticle preparation according to claim 1, wherein the bioabsorbable polymer is a polylactic acid polymer (PLA) or a polylactic acid glycolic acid copolymer (PLGA).

3. The statin-encapsulated nanoparticle preparation according to claim 1, wherein the enhancement of the function of the dental pulp-derived stem cell is at least one of a migratory ability and an ability to produce an angiogenic factor.

4. The statin-encapsulated nanoparticle preparation according to claim 1, wherein the enhancement of the function of the dental pulp-derived stem cell is an increase in expression of HGF.

5. A statin-encapsulated nanoparticle preparation comprising statin-encapsulated nanoparticles in which statin is encapsulated in nanoparticles comprising a bioabsorbable polymer, wherein the statin-encapsulated nanoparticle preparation is for enhancing the therapeutic effect of a cell preparation comprising a dental pulp-derived stem cell for treating ischemic diseases, inflammatory diseases, or neurodegenerative diseases.

6. A statin-encapsulated nanoparticle preparation comprising statin-encapsulated nanoparticles in which statin is encapsulated in nanoparticles comprising a bioabsorbable polymer, wherein the statin-encapsulated nanoparticle preparation is for enhancing the therapeutic effect of a cell preparation comprising a dental pulp-derived stem cell or an adipose-derived stem cell for treating inflammatory diseases or neurodegenerative diseases.

7. A dental pulp-derived stem cell comprising the statin-encapsulated nanoparticle preparation according to claim 1.

8. A cell preparation for treating ischemic diseases, the cell preparation comprising a dental pulp-derived stem cells according to claim 7.

9. A cell preparation for treating inflammatory diseases, the cell preparation comprising a dental pulp-derived stem cell according to claim 7.

10. A cell preparation for treating neurodegenerative diseases, the cell preparation comprising a dental pulp-derived stem cell according to claim 7.

11. The cell preparation according to claim 7 for intravenous administration, arterial administration or topical administration.
